(19) United States
(12) Patent Application Publication
(10) Pub. No.: US 2020/0011855 A1
(43) Pub. Date: Jan. 9, 2020

(54) COMPOSITION FOR PREVENTING OR TREATING LIVER FIBROSIS OR CIRRHOSIS, COMPRISING EXPRESSION OR ACTIVITY ENHANCER OF TIF1y AS ACTIVE INGREDIENT

(71) Applicant: SEOUL NATIONAL UNIVERSITY HOSPITAL, Seoul (KR)

(72) Inventors: Hyo-Soo KIM, Seoul (KR); Eun Ju LEE, Seoul (KR)

(21) Appl. No.: 16/320,154

(22) PCT Filed: Jul. 24, 2017
(86) PCT No.: PCT/KR2017/007929

§ 371 Date: Jul. 2, 2019

(30) Foreign Application Priority Data
Jul. 25, 2016 (KR) 10-2016-0094394

(51) Int. Cl. G01N 33/50; A61K 35/28; A61K 35/54; A61K 38/18

(52) U.S. Cl. CPC G01N 33/5067; A61K 35/28; G01N 2500/10; A61K 38/1833; A61K 35/54

(57) ABSTRACT

The present invention relates to a composition for preventing and treating liver fibrosis or cirrhosis and, more specifically, to a pharmaceutical composition for preventing and treating liver fibrosis or cirrhosis, comprising an expression or activity enhancer of transcriptional intermediary factor 1 gamma (TIF1γ) as an active ingredient, and a method for screening the same. The pharmaceutical composition for preventing or treating liver fibrosis or cirrhosis, comprising an expression or activity enhancer of TIF1γ as an active ingredient, according to the present invention, inhibits the activity of hepatic stellate cells (HSCs) and decreases the expression of α-SMA proteins or the secretion of collagen Type I, thereby ultimately being expected to be developed as a prophylactic or therapeutic agent for liver fibrosis or cirrhosis. In addition, the composition of the present invention is expected to be useful in a method for screening an agent for liver fibrosis or cirrhosis.

### TECHNICAL FIELD

The present invention relates to a composition for preventing and treating liver fibrosis or cirrhosis and, more specifically, to a pharmaceutical composition for preventing or treating liver fibrosis or cirrhosis, comprising an expression or activity enhancer of transcriptional intermediary factor 1 gamma (TIF1γ ) as an active ingredient, and a method for screening the same.

### BACKGROUND ART

Liver fibrosis is a disease in which liver tissue in a chronic inflammatory state is repeatedly damaged and repaired so that connective tissues such as collagen are excessively deposited in the tissue, thereby causing scars in the liver tissue. In general, unlike cirrhosis, liver fibrosis is reversible and in liver fibrosis, thin fibrils appear without nodule formation. Further, once the cause of hepatic injury is eliminated, the liver can be returned to the normal state. However, if the liver fibrosis mechanism is continuously repeated, the liver fibrosis leads to irreversible cirrhosis in which crosslinking between connective tissues increases to accumulate thick fibrils, and a liver lobe loses its normal structure to cause nodule formation.

In addition, cirrhosis refers to a state in which the liver gradually hardens and regenerative nodules of various sizes occur in the liver due to long-lasting hepatocellular damage (hepatitis). Such progressive liver fibrosis leads to cirrhosis and liver failure, requiring liver transplantation as an effective therapy. However, liver transplantation has limitations such as a shortage of organs and long-term immunosuppression. Accordingly, with respect to recent studies on liver fibrosis or cirrhosis treatment, efforts have been made to provide a promising approach for hepatocyte treatment by providing information on cellular and molecular mechanisms such that the demand for liver transplantation may be decreased by reducing liver fibrosis and restoring the function of the liver.

Meanwhile, mesenchymal stem cells are self-inducing cells that may potentially offer a better alternative for cell-based treatment than adult stem cells. Most of the adult stem cells have limitations in clinical application due to lack of available cells and invasive procedures for obtaining cells. However, recently a technology capable of continuously producing, maintaining, and culturing mesenchymal stem cells has been developed, and study results showing that the mesenchymal stem cells are safer from the viewpoint of tumor development and effective for treatment in an animal model (Korean Patent Application Laid-Open No. 10-2010-0074386) have appeared, so that the mesenchymal stem cells will be used as a useful platform for regenerative medicine.

Thus, endogenous and exogenous regeneration of hepatocytes by mesenchymal stem cells is expected to be a promising treatment for alleviating end-stage liver disease and improving liver function and symptoms, but currently, there is a limitation that an accurate mechanism for liver fibrosis or cirrhosis using mesenchymal stem cells has not been clarified.

### DISCLOSURE

### Technical Problem

The present invention has been devised in order to solve the aforementioned problems, and the present inventors confirmed the effect of preventing and treating liver fibrosis or cirrhosis according to the increase in expression of TIF1γ, thereby completing the present invention based on this.

Thus, an object of the present invention is to provide a pharmaceutical composition for preventing or treating liver fibrosis or cirrhosis, comprising an expression or activity enhancer of TIF1γ as an active ingredient.

Further, another object of the present invention is to provide a method for screening a candidate material for preventing or treating liver fibrosis or cirrhosis, the method comprising steps of (1) treating cells or tissue harvested from a patient with liver fibrosis or cirrhosis with a test material and culturing the treated cells or tissues; (2) measuring an expression level of TIF1γ in a cell or tissue culture solution of Step (1); and (3) selecting a candidate material which increases the expression of TIF1γ as compared to a control which is not treated with the test material.

However, a technical problem to be achieved by the present invention is not limited to the aforementioned problem, and other problems that are not mentioned may be clearly understood by a person skilled in the art from the following description.

### Technical Solution

To achieve the object of the present invention as described above, the present invention provides a pharmaceutical composition for preventing or treating liver fibrosis or cirrhosis, comprising an expression or activity enhancer of TIF1γ as an active ingredient.

As an embodiment of the present invention, the expression or activity enhancer of TIF1γ may be human embryonic stem cell-derived mesenchymal stem cells (hE-MSCs).

As another embodiment of the present invention, the composition may downregulate the expression of α-smooth muscle actin (α-SMA) proteins.

As still another embodiment of the present invention, the composition may decrease the secretion of collagen Type I.

Another object of the present invention provides a method for screening a candidate material for preventing or treating liver fibrosis or cirrhosis, the method comprising steps of (1) treating cells or tissues harvested from a patient with liver fibrosis or cirrhosis with a test material and culturing the treated cells or tissues; (2) measuring an expression level of TIF1γ in a cell or tissue culture solution of Step (1); and (3) selecting a candidate material which increases the expression of TIF1γ as compared to a control which is not treated with the test material.

As an embodiment of the present invention, the test material may be a synthetic compound, a microbial culture solution or extract, a synthetic peptide, a nucleic acid, a protein, an antibody, an aptamer, or a natural extract.

Furthermore, the present invention provides a method for preventing or treating liver fibrosis or cirrhosis, the method comprising: administering the pharmaceutical composition to a subject.

In addition, the present invention provides a use of the pharmaceutical composition for preventing or treating liver fibrosis or cirrhosis.

### Advantageous Effects

The pharmaceutical composition for preventing or treating liver fibrosis or cirrhosis, comprising an expression or activity enhancer of TIF1γ as an active ingredient, according to the present invention, inhibits the activity of hepatic stellate cells (HSCs) and decreases the expression of α-SMA proteins or the secretion of collagen Type I, thereby ultimately being expected to be developed as a prophylactic or therapeutic agent for liver fibrosis or cirrhosis. In addition, the composition of the present invention is expected to be useful in a method for screening an agent for liver fibrosis or cirrhosis.

### DESCRIPTION OF DRAWINGS

FIG. 1A illustrates a process for transplanting human embryonic stem cell-derived mesenchymal stem cells into thioacetamide (TAA)-treated mice and confirming a therapeutic effect on liver fibrosis.

FIG. 1B is a set of results of transplanting human embryonic stem cell-derived mesenchymal stem cells into TAA-treated mice and measuring hepatotoxicity indices.

FIG. 1C is a set of results obtained by transplanting human embryonic stem cell-derived mesenchymal stem cells into TAA-treated mice and performing an immunohistochemical analysis using Masson's trichrome (MT) staining.

FIG. 1D is a set of results of confirming that the irregularities on the surface of the liver are restored by transplanting human embryonic stem cell-derived mesenchymal stem cells into TAA-treated mice and performing an immunohistochemical analysis using MT staining.

FIG. 1E is a set of results obtained by transplanting human embryonic stem cell-derived mesenchymal stem cells into TAA-treated mice and performing an immunohistochemical analysis using picrosirius red staining.

FIG. 2A is a result confirming the mRNA expression of α-SMA by performing RT-PCR analysis on hepatic stellate cells after co-culturing human embryonic stem cell-derived mesenchymal stem cells (hE-MSCs) and TGFβ1-activated human hepatic stellate LX2 cells.

FIG. 2B is a result confirming the protein expression of α-SMA by performing Western blot assay on hepatic stellate cells after co-culturing human embryonic stem cell-derived mesenchymal stem cells (hE-MSCs) and TGFβ1-activated human hepatic stellate LX2 cells.

FIG. 2C is a result obtained by performing morphological analysis on hepatic stellate cells after co-culturing human embryonic stem cell-derived mesenchymal stem cells (hE-MSCs) and TGFβ1-activated human hepatic stellate LX2 cells.

FIG. 2D is a result confirming the secretion of collagen Type I by performing enzyme-linked immunosorbent assay on culture fluid of hepatic stellate cells after co-culturing human embryonic stem cell-derived mesenchymal stem cells (hE-MSCs) and TGFβ1-activated human hepatic stellate LX2 cells.

FIG. 3A is a set of results confirming the change in gene expression of 7 anti-fibrosis primary candidate factors of human hepatic stellate LX2 cells according to TGFβ1 treatment by performing RT-PCR analysis.

FIG. 3B is a western blot assay's result confirming the change in protein expression of anti-fibrosis secondary candidate factors TIF1γ, Nm23-H1, and EPLIN of hepatic stellate cells when TGFβ1-activated human hepatic stellate LX2 cells and human embryonic stem cell-derived mesenchymal stem cells (hE-MSCs) are co-cultured.

FIG. 3C is a set of results for selecting TIF1γ as an anti-fibrosis final factor in which an increase in a fibrosis marker, an α-SMA protein, is confirmed during the knockdown of anti-fibrosis secondary candidate factors TIF1γ, Nm23-H1, and EPLIN in human hepatic stellate LX2 cells.

FIG. 3D is a result of confirming a decrease in a fibrosis marker, collagen Type I, in TIF1γ knocked-down human hepatic stellate LX2 cells through enzyme-linked immunosorbent assay.

FIG. 3E is a set of results of confirming a decrease in the mRNA expression and protein expression of α-SMA caused by TIF1γ overexpression of through RT-PCR and Western blot by treating the TIF1γ -overpressing human hepatic stellate LX2 cells with TGFβ1 in order to verify the anti-fibrosis function. FIG. 4A is a set of results of confirming the secretion of hepatocyte growth factor (HGF) from human embryonic stem cell-derived mesenchymal stem cells (hE-MSCs) by performing enzyme-linked immunosorbent assay.

FIG. 4B is a result of confirming an increase in the expression of TIF1γ caused by HGF by adding human recombinant HGF to a TGFβ1-activated human hepatic stellate LX2 cell line and performing Western blot assay on TIF1γ and α-SMA.

FIG. 4C is a Western blot result of confirming an effect of HGF on the expression of TIF1γ through the knockdown of HGF secreted from human embryonic stem cell-derived mesenchymal stem cells (hE-MSCs). It can be confirmed that when HGF is decreased, TIF1γ is decreased and α-SMA is increased.

FIG. 5A is a result of confirming that TIF1γ is expressed at human hepatic stellate cell positions in a normal mouse liver through an immunohistochemical analysis.

FIG. 5B is a set of results obtained by transplanting human embryonic stem cell-derived mesenchymal stem cells into TAA-treated mice and performing an immunohistochemical analysis in order to confirm a change in the expression of TIF1γ.

FIG. 5C is a set of results obtained by transplanting human embryonic stem cell-derived mesenchymal stem cells into TAA-treated mice and quantitatively analyzing TIF1γ positive cell numbers in order to confirm a change in the expression of TIF1γ. It was confirmed that the TIF1γ positive cell numbers were decreased in mouse liver tissue treated by TAA whereas the TIF1γ positive cell numbers were increased in human embryonic stem cell-derived mesenchymal stem cell-transplanted liver tissue.

FIG. 5D is a set of results obtained by transplanting human embryonic stem cell-derived mesenchymal stem cells into TAA-treated mice and performing Western blot assay in order to confirm a change in the expression of TIF1γ. It was confirmed that the expression of TIF1γ was decreased in mouse liver tissue treated by TAA whereas the expression of TIF1γ was increased in human embryonic stem cell-derived mesenchymal stem cell-transplanted liver tissue.

FIG. 6A illustrates an experimental process for confirming the differentiation of hepatic stellate cells (HSCs) and the secretion of human hepatocyte growth factor (hHGF) according to the transplantation of human embryonic stem cell-derived mesenchymal stem cells (hE-MSCs).

FIG. 6B is a result obtained by performing an immunohistochemical analysis using tissue after transplanting human embryonic stem cell-derived mesenchymal stem cells (hE-MSCs) labeled with a fluorescent dye.

FIG. 6C is a set of results obtained by performing an immunohistochemical analysis in order to confirm the differentiation of hepatic stellate cells (HSCs) according to the transplantation of human embryonic stem cell-derived mesenchymal stem cells (hE-MSCs). (CRBP 1: hepatic stellate cell marker, Hepatocyte: hepatic cell marker)

FIG. 6D is a set of immunohistochemical analysis results confirming the secretion of human hepatocyte growth factor (hHGF) according to the transplantation of human embryonic stem cell-derived mesenchymal stem cells (hE-MSCs) using a human hepatocyte growth factor-specific antibody.

FIG. 7A is a result of confirming a decrease in the expression of TIF1γ in cirrhotic tissue by performing an immunohistochemical analysis on human normal liver tissue and human cirrhotic liver tissue.

FIG. 7B is a result of confirming a decrease in the expression of TIF1γ together with an increase in the expression of α-SMA in cirrhotic tissue by performing an immunohistochemical analysis on human normal liver tissue and human cirrhotic liver tissue.

### MODES OF THE INVENTION

It was confirmed that a composition according to the present invention has an effect of preventing or treating liver fibrosis or cirrhosis by comprising an expression or activity enhancer of TIF1γ as an active ingredient, inhibiting the activity of hepatic stellate cells (HSCs), and promoting the secretion of hepatocyte growth factor (HGF), thereby completing the present invention based on these facts. Hereinafter, the present invention will be described in detail.

The present invention provides a pharmaceutical composition for preventing or treating liver fibrosis or cirrhosis, comprising an expression or activity enhancer of TIF1γ as an active ingredient.

The term “prevention” used in the present invention refers to all actions that inhibit liver fibrosis or cirrhosis or delay the onset of liver fibrosis or cirrhosis by administering the pharmaceutical composition according to the present invention.

The term “treatment” used in the present invention refers to all actions that ameliorate or beneficially change symptoms of liver fibrosis or cirrhosis by administering the pharmaceutical composition according to the present invention.

Liver fibrosis which is a disease to be prevented or treated by the composition of the present invention refers to a disease in which liver tissue in a chronic inflammatory state is repeatedly damaged and repaired so that connective tissues such as collagen are excessively deposited in the tissue, thereby causing scars in the liver tissue. In general, unlike cirrhosis, liver fibrosis is reversible and in liver fibrosis, thin fibrils appear without nodule formation. Further, once the cause of hepatic injury is eliminated, the liver can be returned to the normal state. However, if the liver fibrosis mechanism is continuously repeated, the liver fibrosis leads to irreversible cirrhosis in which crosslinking between connective tissues increases to accumulate thick fibrils, and a liver lobe loses its normal structure to cause nodule formation.

In addition, cirrhosis which is a disease to be prevented or treated by the composition of the present invention refers to a state in which the liver gradually hardens and regenerative nodules of various sizes occur in the liver due to long-lasting hepatocellular damage (hepatitis).

The “transcriptional intermediary factor 1 gamma (TIF1γ)” used in the present invention is a gene that is also known as tripartite motif-containing 33 (TRIM33) which is a transcriptional factor involved in cell differentiation and development.

In the present invention, the expression or activity of TIF1γ is decreased by a fibrosis signal such as thioacetamide (TAA) or transforming growth factor beta 1 (TGFβ1).

The expression or activity enhancer of TIF1γ may be hepatocyte growth factor (HGF), a histone deacetylase (HDAC) inhibitor, a transforming growth factor beta (TGF-β) signal inhibitor, or an epithelial-mesenchymal transition (EMT) inhibitor, but is not limited to the types described above.

The term “mesenchymal stem cell (MSC)” in the present invention, as a stem cell isolated from bone marrow, blood, the dermis, the periosteum, and the like, refers to a pluripotent or multipotent cell that may be differentiated into various cells, for example, adipocytes, chondrocytes, osteocytes, and the like. In particular, the mesenchymal stem cell in the present invention may be an animal mesenchymal stem cell, preferably a mammalian mesenchymal stem cell, more preferably a human mesenchymal stem cell. Further, the mesenchymal stem cell of the present invention may be derived from bone marrow, adipocyte tissue, peripheral blood, the liver, the lungs, amniotic fluid, the placental chorion or umbilical cord blood, but is not limited thereto.

In addition, in the present invention, the expression or activity enhancer of TIF1γ may downregulate the expression of α-SMA proteins or decrease the secretion of collagen Type I.

As another aspect of the present invention, the present invention provides a method for screening a candidate material for preventing or treating liver fibrosis or cirrhosis. More specifically, the method of the present invention may comprise steps of (1) treating cells or tissues harvested from a patient with liver fibrosis or cirrhosis with a test material and culturing the treated cells or tissues; (2) measuring an expression level of TIF1γ in a cell or tissue culture solution of Step (1); and (3) selecting a candidate material which increases the expression of TIF1γ as compared to a control which is not treated with the test material, but is not limited thereto.

In the screening method of the present invention, the test material may comprise a synthetic compound, a microbial culture solution or extract, a synthetic peptide, a nucleic acid, a protein, an antibody, an aptamer, or a natural extract, but is not limited thereto, and any material may be used as long as the test material has an effect of increasing the expression of TIF1γ.

In an embodiment of the present invention, in order to confirm the therapeutic effect of TIF1γ on liver fibrosis or cirrhosis, the inhibitory effect of human embryonic cell-derived mesenchymal stem cells (hE-MSCs) on liver fibrosis or cirrhosis of mice was confirmed by culturing human embryonic stem cell-derived mesenchymal stem cells (hE-MSCs)(see Example 1) and inducing liver fibrosis in mice with thioacetamide (TAA)(see Example 2), and the inhibitory effect of hE-MSCs on the activity of human hepatic stellate cells was confirmed by confirming the expression degree of α-SMA and performing a morphological analysis and enzyme-linked immunosorbent assay after co-culturing human embryonic stem cell-derived mesenchymal stem cells (hE-MSCs) and TGFβ1-activated human hepatic stellate LX2 cells (see Example 3).

In another embodiment of the present invention, the inhibitory effect of TIF1γ on the activity of human hepatic stellate LX2 cells was confirmed by expression degree, and performing functional analysis and enzyme-linked immunosorbent assay on anti-fibrosis candidate factors in human hepatic stellate LX2 cells (see Example 4). In still another embodiment of the present invention, it was confirmed that the expression of TIF1γ is increased by hepatocyte growth factor (HGF) by performing enzyme-linked immunosorbent assay and Western blot assay on the HGF in human embryonic stem cell-derived mesenchymal stem cells (hE-MSCs) (see Example 5).

In yet another embodiment of the present invention, the effects of transplantation of human embryonic stem cell-derived mesenchymal stem cells (hE-MSCs) on TAA-treated liver fibrotic mice were confirmed (see Example 6), the differentiation of human stellate cells (HSCs) and the secretion of human hepatocyte growth factor (hHGF) according to the transplantation of human embryonic stem cell-derived mesenchymal stem cells were confirmed (see Example 7), and a TIF1γ reduction effect in a human cirrhotic liver was confirmed (see Example 8). Accordingly, the pharmaceutical composition for preventing or treating liver fibrosis or cirrhosis, comprising an expression or activity enhancer of TIF1γ as an active ingredient, according to the present invention, inhibits the activity of hepatic stellate cells (HSCs) and decreases the expression of α-SMA proteins or the secretion of collagen Type I, and thus has an effect of preventing or treating liver fibrosis or cirrhosis.

The pharmaceutical composition according to the present invention may comprise a pharmaceutically acceptable carrier in addition to the active ingredient. In this case, the pharmaceutically acceptable carrier is typically used during formulation, and includes, but is not limited to, lactose, dextrose, sucrose, sorbitol, mannitol, starch, gum acacia, calcium phosphate, alginate, gelatin, calcium silicate, microcrystalline cellulose, polyvinyl pyrrolidinone, cellulose, water, syrup, methylcellulose, methyl hydroxybenzoate, propyl hydroxybenzoate, talc, magnesium stearate, mineral oil, and the like. Furthermore, the pharmaceutically acceptable carrier may further include a lubricant, a wetting agent, a sweetening agent, a flavoring agent, an emulsifier, a suspension, a preservative, and the like, in addition to the aforementioned ingredients.

The pharmaceutical composition of the present invention may be orally administered or may be parenterally administered (for example, administered intravenously, subcutaneously, intraperitoneally, or topically), and although the administration dose may vary depending on a patient's condition and body weight, severity of disease, drug form, and administration route and period, it may be properly selected by the person skilled in the art.

The pharmaceutical composition of the present invention is administered in a pharmaceutically effective amount. In the present invention, “pharmaceutically effective amount” means an amount sufficient to treat diseases at a reasonable benefit/risk ratio applicable to medical treatment, and an effective dosage level may be determined according to factors including type of disease of patients, the severity of disease, the activity of drugs, sensitivity to drugs, administration time, administration route, excretion rate, treatment period, and simultaneously used drugs, and other factors well known in the medical field. The pharmaceutical composition according to the present invention may be administered as an individual therapeutic agent or in combination with other therapeutic agents, may be administered sequentially or simultaneously with conventional therapeutic agents, and may be administered in a single dose or multiple doses. It is important to administer the composition in a minimum amount that can obtain the maximum effect without any side effects, in consideration of all the aforementioned factors, and this amount may be easily determined by one skilled in the art.

Specifically, an effective amount of the pharmaceutical composition of the present invention may vary depending on the age, sex, condition, and body weight of a patient, the absorption of the active ingredients in the body, inactivation rate and excretion rate, disease type, and the drugs used in combination, and in general, 0.001 to 150 mg, preferably 0.01 to 100 mg of the pharmaceutical composition of the present invention per 1 kg of a body weight may be administered daily or every other day or may be administered once or divided into two to three times a day. However, since the effective amount may be increased or decreased depending on the administration route, the severity of obesity, the sex, the body weight, the age, and the like, the administration dose is not intended to limit the scope of the present invention in any way.

Furthermore, the present invention provides a method for preventing or treating liver fibrosis or cirrhosis, the method comprising: administering the pharmaceutical composition to a subject.

The “subject” as used herein refers to a target in need of treatment of a disease, and more specifically, refers to a mammal such as a human or a non-human primate, a mouse, a rat, a dog, a cat, a horse, and a cow.

Hereinafter, preferred Examples for helping the understanding of the present invention will be suggested. However, the following Examples are provided only to more easily understand the present invention, and the contents of the present invention are not limited by the following Examples.

### EXAMPLES

### Example 1

### Experimental Preparation

1-1. Culture of Human Embryonic Stem Cell-Derived Mesenchymal Stem Cells (hE-MSCs)

Research related to the present invention was approved by the Medical Research Ethics Committee of the Seoul National University Hospital. The SNUhES3 hESCs, which are an embryonic stem cell line, were cultured in a Petri dish for 14 days in order to form embryonic bodies without fibroblast growth factor-2 (FGF-2). Thereafter, after the cultured embryonic bodies were attached to a gelatin-coated dish, the cultured embryonic bodies were cultured in a medium in which 10% fetal bovine serum (FBS; Invitrogen) was added to low-glucose DMEM (Invitrogen) for 16 days, and then differentiated cells were proliferatively cultured in an EGM-2 mV medium (Lonza). The differentiation of proliferatively cultured cells into adipocytes, osteocytes, myocytes, and chondrocytes was tested under appropriate conditions in order to evaluate the differentiation potential of the proliferatively cultured cells into mesenchymal stem cells. Human embryonic stem cell-derived mesenchymal stem cells (hE-MSCs) were obtained by the method, and in vitro and in vivo experiments were performed using human embryonic cell-derived mesenchymal stem cells (hE-MSCs) subcultured 13-14 generations.

1-2. Statistical Analysis

A statistical analysis was performed using GraphPad Prism 6 software (GraphPad Software, La Jolla, Calif., USA). The result values were expressed as mean±standard error of the mean (SEM), the deviations between respective groups were compared by a t-test, and it was determined that P<0.05 was a statistically significant result.

### Example 2

### Confirmation of Inhibitory Effect of Human Embryonic Stem Cell-Derived Mesenchymal Stem Cells (hE-MSCs) on Mouse Liver Fibrosis

2-1. Preparation of Tthioacetamide (TAA)-Treated Liver Fibrotic Mice

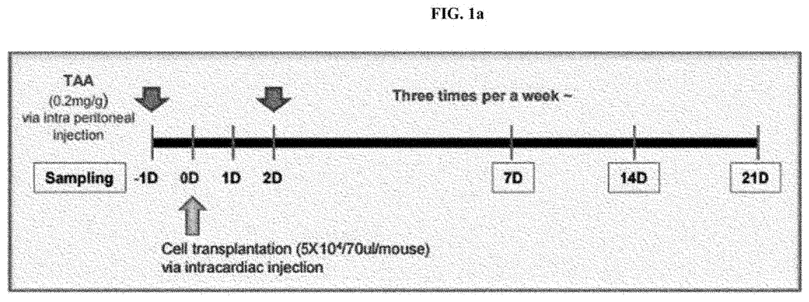

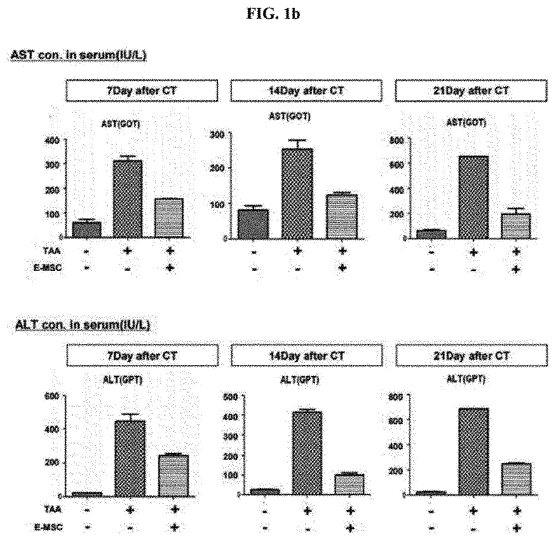

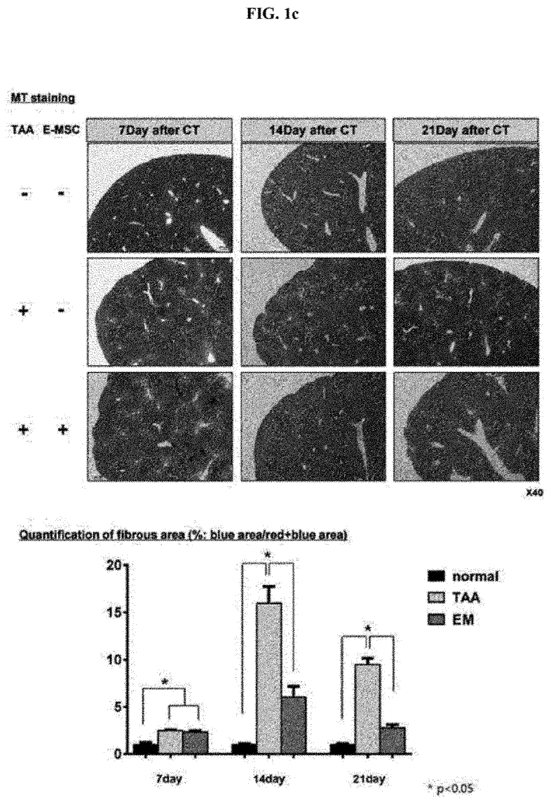

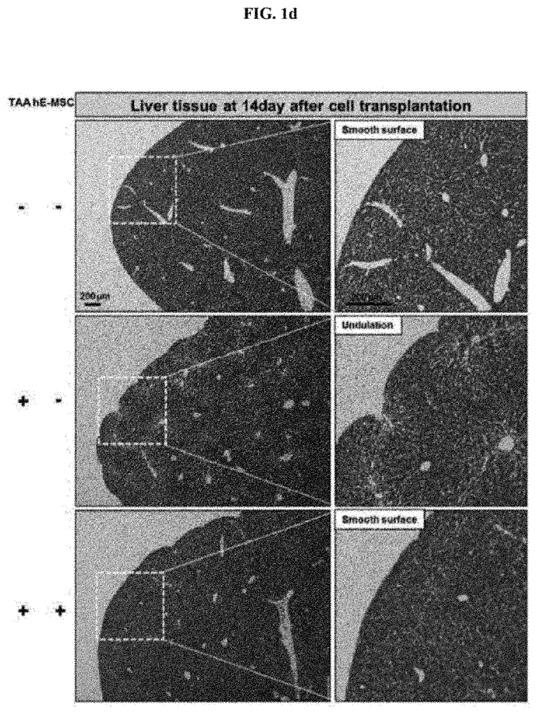

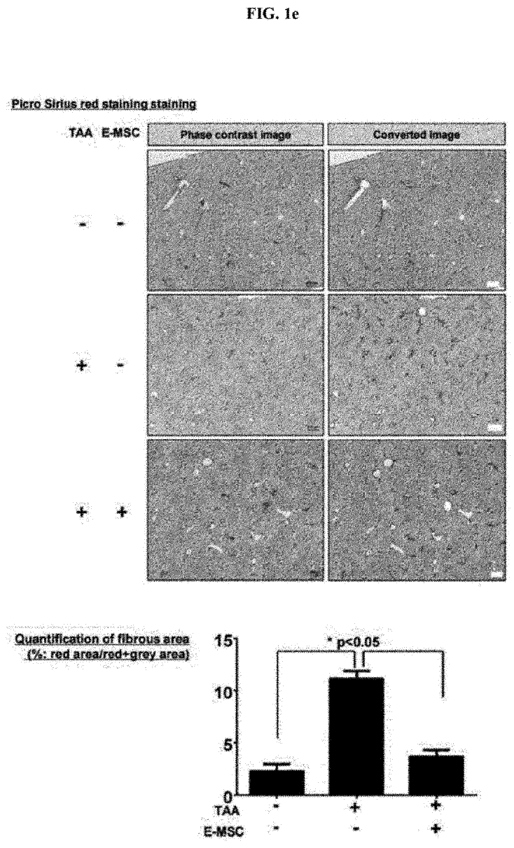

As illustrated in FIG. 1A, human embryonic stem cell-derived mesenchymal stem cells (hE-MSCs) were transplanted into immunodeficient mice treated with thioacetamide (TAA), and confirmation of a potential therapeutic effect on liver fibrosis was attempted. Guidelines on experimentation animal breeding, use, treatment, and management of all animals and all animal research protocols were approved by the Institutional Animal Care and Use Committee (IACUC) of the Seoul National University Hospital. In order to prepare a TAA-treated liver fibrotic mouse model, 200 mg/kg of thioacetamide (TAA; Sigma Aldrich, St. Louis, Mo., USA) or phosphate buffered saline (PBS) as a control was injected into 12 to 13-week old male BALB/c-nu mice with a body weight of 20 to 25 g via intraperitoneal injection three times a week for 1 to 3 weeks. The TAA-treated mice were randomly divided into two groups administered either human embryonic cell-derived mesenchymal stem cells (hE-MSCs) or PBS. BALB/c-nu mice were intraperitoneally anesthetized using Zoletil (Virbac, France) and Rompun (Bayer, Germany) 24 hours after the injection of TAA into the BALB/c-nu mice, and then 5×10⁴ of hE-MSCs were injected intracardiacally into the mice, and a total volume of 70 p1 of PBS as a control was injected into the mice using 31-G insulin syringes (BD, San Jose, Calif., USA). In order to track the transplanted cells (hE-MSCs), the hE-MSCs were labeled with CellTracker™ CM-DII (Invitrogen) before transplantation, and a growth medium at a concentration of 4 μg/ml was added thereto at 37° C. for 24 hours. The hE-MSCs were injected intracardiacally into the mice, the mice recovered for 2 days, and thereafter, TAA was continuously injected three times a week.

2-2. Serum Assays

In order to confirm hepatotoxicity indices according to the transplantation of hE-MSCs from the mice prepared by the method in Example 2-1, blood samples were collected from the hearts of the anesthetized mice on each of Day 7, Day 15, and Day 21 after the cell transplantation of hE-MSCs. Sera were centrifuged at 3,000 rpm for 15 minutes, and stored at 80° C. until analysis. In order to test liver function, activities of aspartate aminotransferase (AST) and alanine aminotransferase (ALT) were measured according to the manufacturer's instruction using an automatic chemistry analyzer (HITACHI 7070).

As a result, as illustrated in FIG. 1B, it was confirmed that, 7 days after transplantation, hepatotoxicity indices were downregulated by measuring the activities of AST and ALT which are hepatocyte enzymes in the TAA-treated group and transplanted with human embryonic stem cell-derived mesenchymal stem cells (hE-MSCs), and that this effect was maintained on Day 14 and Day 21 after the transplantation of hE-MSCs.

2-3. Immunohistochemical Analysis

After blood was collected from the mice described in Example 2-2, the livers of the mice were removed through perfusion with cold PBS in order to perform an immunohistochemical analysis for evaluation of the therapeutic effect of hE-MSCs on liver fibrosis. The liver was fixed with a 10% neutral formalin solution and paraffin, and cut to a thickness of 4 to 5 μm. Paraffin sections were subjected to hematoxylin and eosin, MT or picrosirius red staining according to standard protocol. Masson's trichrome (MT) and picrosirius red staining were used to detect collagen and visualize connective tissues. An image was obtained using a Leica optical microscope (Leica, Wetzlar, Germany). A quantitative image analysis of a fibrotic liver area and an MT staining and picrosirius red staining area was measured using SABIA software (Metoosoft, Seoul, Korea) and ImageJ software (National Institutes of Health; Bethesda, Md., USA).

As a result, as illustrated in FIGS. 1C and 1D, a histological analysis of collagen fiber from a group treated with hE-MSCs was performed using an MT staining method, and 7 days after transplantation, it was confirmed that the fibrotic area was decreased, but the difference was not significant, and it was confirmed that in a hepatic injury induced by TAA, recovery rapidly proceeded and the undulations of the surface of the liver were restored.

Further, as illustrated in FIG. 1E, as a result of visualization through picrosirius red staining that detects Types I and III collagen in order to confirm the degree of collagen in the tissue on Day 14 after the treatment with hE-MSCs, the therapeutic effect of hE-MSCs on liver fibrosis was confirmed.

### Example 3

### Confirmation of Inhibitory Effect of Human Embryonic Cell-Derived Mesenchymal Stem Cells (hE-MSCs) on Activity of Human Hepatic Stellate Cells

3-1. Co-Culture of Cells

A human hepatic stellate cell line LX2 was obtained from Dr. Friedman, and cultured under a 5% CO₂ humidified culture condition and at a temperature of 37° C. in a high-glucose DMEM of GlutaMax (Gibco, Grand Island, N.Y., USA), 5% or 10% FBS and 1% (v/v) penicillin/streptomycin (Gibco, LX2 complete medium). Thereafter, in order to evaluate the therapeutic effect of hE-MSCs on liver fibrosis, hE-MSCs and a TGFβ1-activated human stellate cell line (LX2 cell line) were co-cultured in vitro as follows.

After the LX2 cells (2×10⁵ cells/ml) were plated onto a 10-cm Petri dish, the cells were cultured for 2 to 3 days until 50% confluence, and then the cell medium was replaced with 0.5% FBS. The LX2 cells were treated with 5 ng/ml of recombinant human TGFβ1 (R&D Systems, Minneapolis, Md., USA) daily for 4 days. Whenever replaced, the medium was treated with a cytokine. LX2 cells pre-treated with hTGFβ1 were co-cultured with 8×10⁵ hE-MSCs in 5 ng/ml of hTGFβ1 and 0.5% FBS per dish in a Transwell insert (0.4-nm pore size, Corning, Corning, N.Y., USA).

3-2. Real-Time PCR Analysis

Smooth muscle actin (α-SMA) is generally a liver fibrosis marker induced in activated hepatic stellate cells. In order to evaluate the degree of liver fibrosis, the expression amount of α-SMA mRNA was evaluated. All the RNAs were isolated from cultured cells according to the manufacturer's instruction using the QIAshredder and RNeasy plus mini kit (Qiagen, Venlo, Netherlands). cDNA was synthesized from 1 μg of RNA using the PrimeScript 1st strand cDNA Synthesis Kit (Takara, Tokyo, Japan). Real-time PCR was performed using the Power SYBR Green PCR master mix (Applied Biosystems, Foster City, Calif., USA) in an apparatus of the ABI PRISM-7500 sequence detection system (Applied Biosystems). Glyceraldehyde 3-phosphate dehydrogenase (GAPDH) was used as an internal control in order to calculate a relative change in gene expression. A real-time PCR primer was designed using Primer3 software (Whitehead Institute/MIT Center for Genome Research) and synthesized by Bioneer (Seoul, Korea). The used α-SMA is shown in the following Table 1.

TABLE 1

| primer | | sequence |
|---|---|---|
| α-SMA | Forward | 5′ GGCAAGTGATCACCATCGGA 3′ |
| | Reverse | 5′ TCTCCTTCTGCATTCGGTCG 3′ |

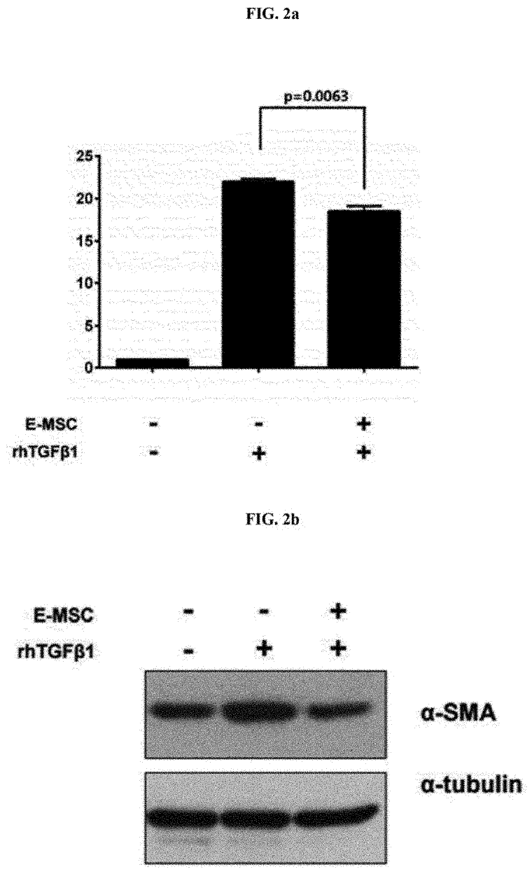

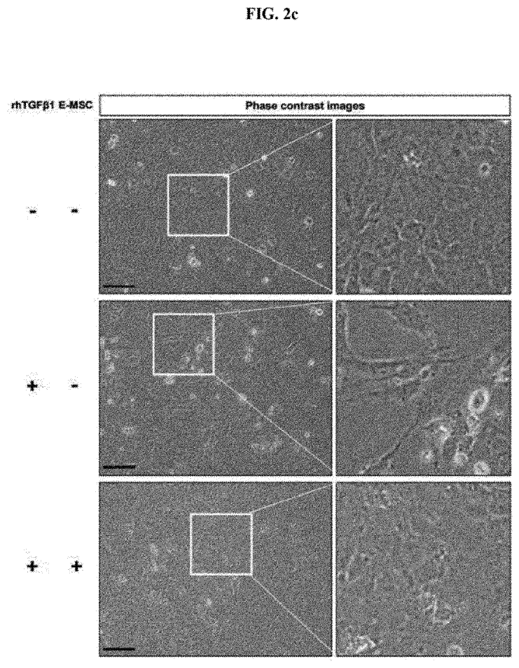

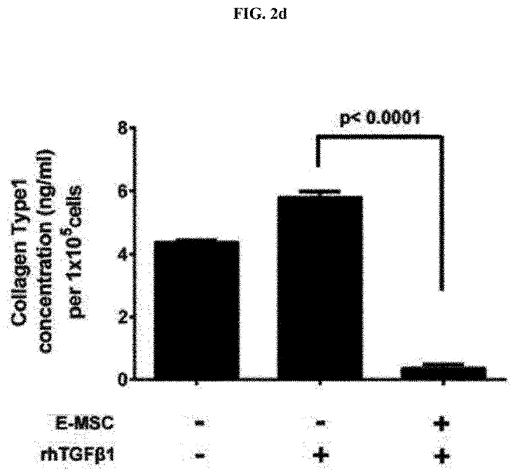

As a result, as illustrated in FIG. 2A, it was confirmed that, after hE-MSCs and the TGFγ1-activated human hepatic stellate cell line (LX2 cell line) were co-cultured, the mRNA expression of α-SMA was downregulated in LX2 cells.

3-3. Western Blot Assay

In order to evaluate the degree of liver fibrosis, the expression amount of α-SMA proteins was evaluated by a Western blot assay method. The cultured cell or tissue sample was dissolved in a protein lysis buffer (0.1% sodium dodecyl sulfate [SDS] comprising 50 mM Tris-HCl, 150 mM NaCl, 0.5% deoxycholate, 1% NP40, and a protease inhibitor cocktail [Roche, Indianapolis, Ind., USA]). After the whole protein extract (2,530 μg) was boiled at 95° for 5 minutes, the extract was isolated by SDS-PAGE, and then transferred to polyvinylidene fluoride membranes (Millipore, Darmstadt, Germany) using a BioRad transfer unit (BioRad, Hercules, Calif., USA). The cell membrane was blocked with 5% skim milk diluted in Tris-buffered saline (TBS) including 0.1% Tween-20 and cultured with α-SMA (1:3000) antibodies, and an anti-α-tubulin antibody (1:5000, Sigma-Aldrich) or an anti-GAPDH antibody (1:30,000, Sigma-Aldrich) was used as an internal control. After the cell membrane was washed, the washed cell membrane was cultured with horseradish peroxidase-conjugated secondary antibodies, and an immune response was confirmed after washing to quantify the cell membrane using TINA 2.0 (RayTest) or the ImageJ (National Institutes of Health) program.

As a result, as illustrated in FIG. 2B, after hE-MSCs and the TGFβ1-activated human hepatic stellate cell line (LX2 cell line) were co-cultured, it was confirmed that the expression levels of α-SMA proteins were all downregulated.

3-4. Morphological Analysis of Cells

The cells co-cultured by the method in Example 3-1 were observed under a phase contrast microscope, and images were captured.

As a result, as illustrated in FIG. 2C, the morphological change related to liver fibrosis in liver stellate cells treated with TGFβ1 was decreased by co-culture with hE-MSCs.

3-5. Enzyme-Linked Immunosorbent Assay (ELISA)

In order to confirm the secretion of collagen Type I and a cytokine in a culture supernatant of cells cultured in Example 3-1, the analysis was performed according to the manufacturer's protocol using the ELISA kit (Cusabio Biotech Co., China). Measurement was made using the Multiskan GO microplate spectrophotometer (Thermo Scientific, Waltham, Mass., USA).

As a result, as illustrated in FIG. 2D, the secretion of collagen Type I was generally upregulated in the fibrotic liver, and was decreased in LX2 cells co-cultured with hE-MSCs. As a result of the co-culture experiment in Example 3, it was confirmed that hE-MSCs inhibited the activity of human hepatic stellate cells.

### Example 4

### Confirmation of Inhibitory Effect of TIF1γ on Activity of Human Hepatic Stellate Cells (LX2 Cells)

4-1. Real-Time PCR Analysis

In order to confirm the mechanism in which hE-MSCs inhibit the activity of hepatic stellate cells, we analyzed the expression of anti-fibrosis candidate factors in hepatic stellate cells. Since activated hepatic stellate cells induce a mesenchymal-epithelial transition as a precursor phenomenon of fibrosis, 7 genes shown in the following Table 2 were selected as a negative regulator of the mesenchymal-epithelial transition. Real-time PCR analysis was performed by the method described in Example 3-2, and a real-time PCR primer was designed using Primer3 software (Whitehead Institute/MIT Center for Genome Research) and synthesized by Bioneer (Seoul, Korea). The primers of the used anti-fibrosis candidate factors are shown in the following Table 2.

TABLE 2

| primer | | sequence |
|---|---|---|
| TIF1γ | Forward | 5′ CTCCGGGATCATCAGGTTTA 3′ |
| | Reverse | 5′ ACTGCTCAACATGCAAGCAC 3′ |
| Nm23- | Forward | 5′ GCCTGGTGAAATACATGCAC 3′ |
| H1 | Reverse | 5′ AGTTCCTCAGGGTGAAACCA 3′ |
| EPLIN | Forward | 5′ CTGCGTGGAATGTCAGAAGA 3′ |
| | Reverse | 5′ TTTTGCTTGCCCATAGATCC 3′ |
| KLF17 | Forward | 5′ GTCCCAGTCATTGCTGGTTT 3′ |
| | Reverse | 5′ TGGGAGCGTTTGGTATAAGC 3′ |
| PIAS1 | Forward | 5′ CATCGCCATTACTCCCTGTT 3′ |
| | Reverse | 5′ AAGCGCTGACTGTTGTCTGA 3′ |
| ALR | Forward | 5′ CCTGTGAGGAGTGTGCTGAA 3′ |
| | Reverse | 5′TCCACTTTTGAGCAGTCGAA 3′ |
| MBNL1 | Forward | 5′ CAGCCGCCTTTAATCCCTAT 3′ |
| | Reverse | 5′ TGTCAGCAGGATGAGCAAAC 3′ |

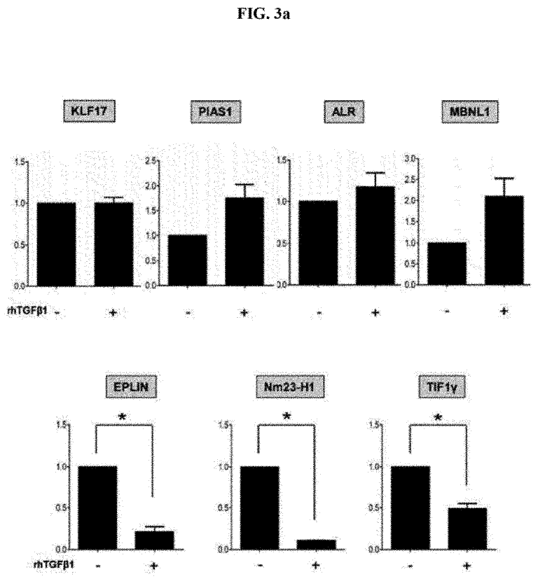

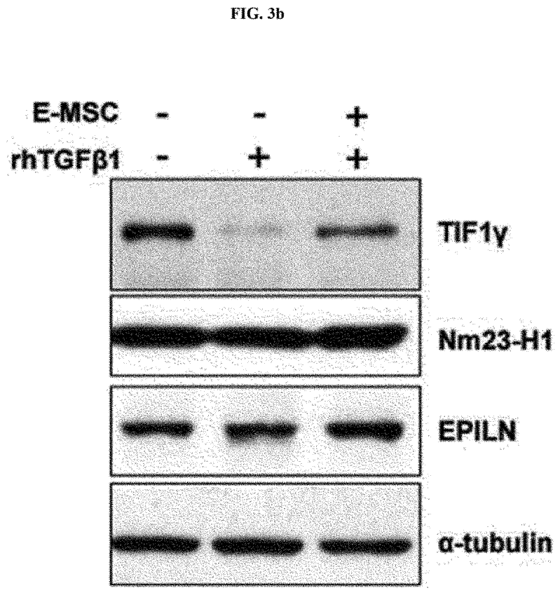

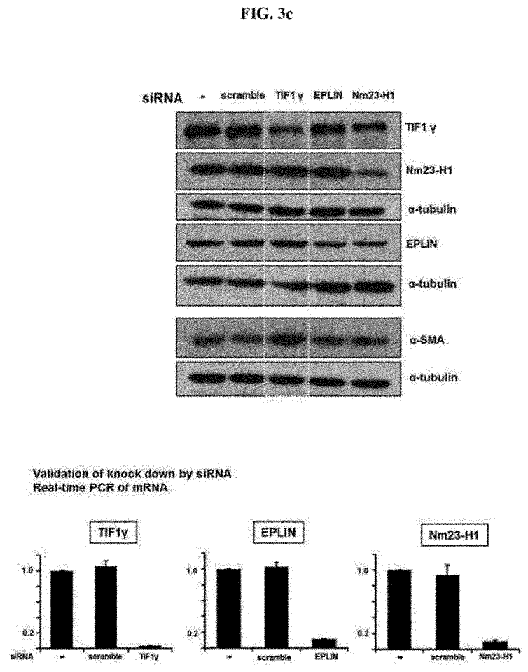

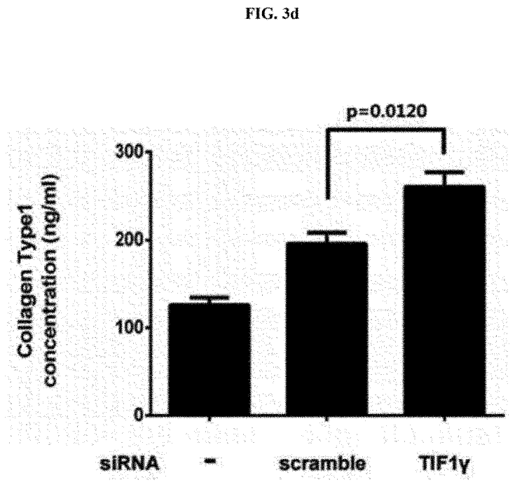

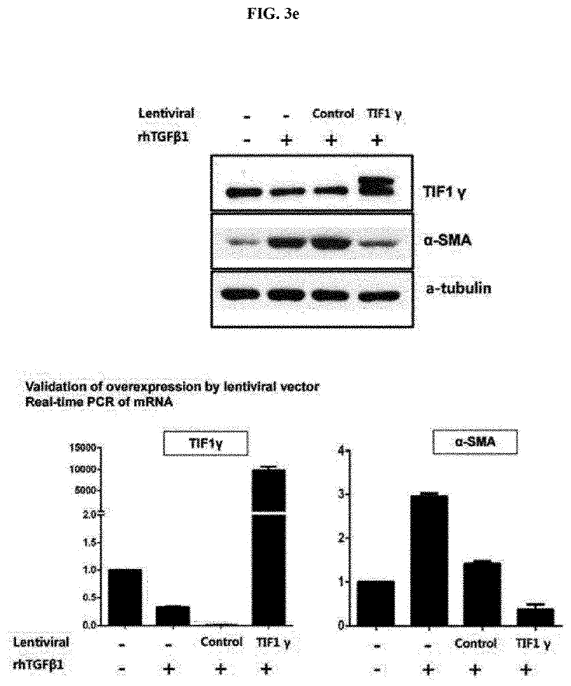

As a result, as illustrated in FIG. 3A, EPLIN encoding cytoskeletal proteins that inhibit actin filament depolymerization, nucleoside diphosphate kinase A (Nm23-H1) which is a metastasis suppressor, and TIF1γ were downregulated in LX2 cells treated with TGFβ1.

4-2. Western Blot Assay

In order to confirm the degree of protein expression of EPLIN, Nm23-H1, and TIF1γ which were selected as anti-fibrosis candidate factors in Example 4-1, Western blot assay was performed. LX2 cells were cultured using TIF1γ (1:1000), EPLIN (1:500, Abcam), and anti-Nm23-H1(1:1000, Santa Cruz Biotechnology) antibodies, and an anti-α-tubulin antibody (1:5000, Sigma-Aldrich) or an anti-GAPDH antibody (1:30,000, Sigma-Aldrich) was used as an internal control.

As a result, as illustrated in FIG. 3B, only TIF1γ was downregulated in LX2 cells treated with TGFβ1, and upregulated when co-cultured with hE-MSCs, whereas EPLIN and Nm23-H1 did not cause any change.

4-3. Loss and Gain of Function Analysis

An RT-PCR assay for loss and gain of function was performed to verify the function of TIF1γ. The loss of function in LX2 cells was analyzed using TIF1γ, EPLIN, Nm23-H1-specific siRNA, and Matafectene-pro as a control siRNA (Santa Cruz Biotechnology). 7 hours later, the medium was replaced with a fresh complete medium, and the cells were cultured for 1 to 4 days without any replacement of medium. The gain of function was used by transfecting a pCMV-TIF1γcDNA vector with Matafectene-pro in the LX2 cells. 7 hours later, the medium was replaced with a fresh complete LX2 medium, and from the next day, the medium was replaced by adding 5 ng/ml of hTGFβ1 every 24 hours, followed by sampling 48 hours or 96 hours later.

As a result, as illustrated in FIG. 3C, it was confirmed that the upregulation of α-SMA was observed by western blot analysis when the expression of TIF1γ in LX2 cells was knocked down by siRNA, whereas the knockdown of EPLIN or Nm23-H1 did not affect the expression of α-SMA. The knockdown of each gene was confirmed by RT-PCR of mRNA.

Furthermore, as illustrated in FIG. 3D, as a result of performing enzyme-linked immunosorbent assay, the knockdown of TIF1γ induced an increase in secretion of collagen Type I.

Likewise, as illustrated in FIG. 3E, as a result of performing RT-PCR and Western blot analysis, the overexpression of TIF1γ decreased the expression of α-SMA in LX2 cells by TGFβ1. Accordingly, as a result of Example 4, it was confirmed that the anti-fibrosis activity of hE-MSCs was related to TIF1 γ upregulation in hepatic stellate cells, and from this, it can be inferred that TIF1γ is a novel anti-fibrosis factor.

### Example 5

### Confirmation of TIF1γ Upregulation Effect of Hepatocyte Growth Factor (HGF) in Human Embryonic Cell-Derived Mesenchymal Stem Cells (hE-MSCs)

5-1. Enzyme-Linked Immunosorbent Assay (ELISA)

In order to see the relationship between TIF1γ upregulation and the activity of hE-MSCs, HGF, VEGF, and FGF-2 known as representative cytokines of mesenchymal stem cells were identified from a hE-MSC culture solution by the enzyme-linked immunosorbent assay method described in Example 3-5.

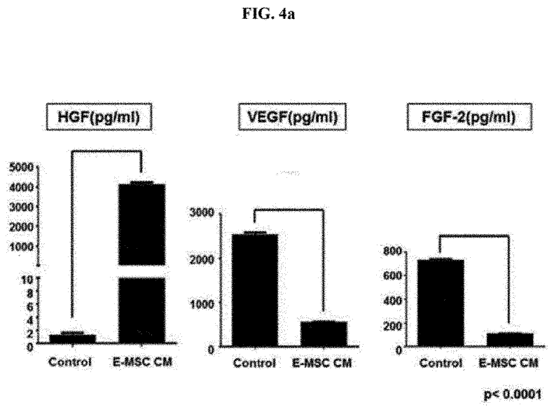

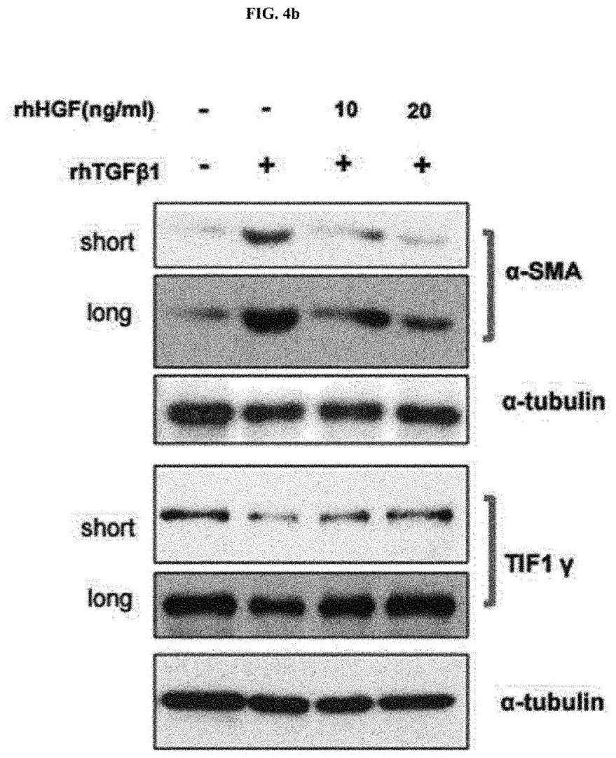

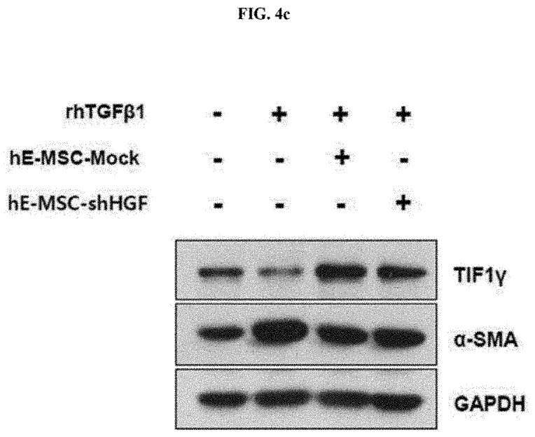

As a result, as illustrated in FIG. 4A, it was observed that hE-MSCs relatively increased the secretion of hepatocyte growth factor (HGF) when compared with the control.

5-2. Western blot assay

In order to confirm the effect of hepatocyte growth factor (HGF) on the expression of TIF1γ in LX2 cells, the expression of α-SMA and TIF1γ was confirmed by Western blot analysis by culturing LX2 cells treated with TGFβ1 together with recombinant hHGF. Further, HGF-specifically knocked down hE-MSCs were prepared by shRNA (sequence: ACCATTTGGAATGGAATTCCA), and it was confirmed whether hepatocyte growth factor (HGF) regulated the expression of TIF1γ in human hepatic stellate cells by co-culturing the hE-MSCs and LX2 cells by the method described in Example 3-1.

As a result, as illustrated in FIG. 4B, in LX2 cells treated with TGFβ1, HGF downregulated the expression of α-SMA, whereas HGF upregulated the level of TIF1γ. Likewise, as illustrated in FIG. 4C, it was confirmed that HGF upregulated α-SMA in knocked down hE-MSCs.

### Example 6

### Confirmation of Effect of Transplantation of Human Embryonic Cell-Derived Mesenchymal Stem Cells (hE-MSCs) on TAA-Treated Liver Fibrotic Mice

6-1. Immunohistochemical analysis In order to confirm the level of TIF1γ of TAA-treated mouse livers experiencing liver fibrosis, the level of TIF1γ was analyzed by an immunohistochemistry technique using the method described in Example 2-3. In order to confirm the expression of TIF1γ in the livers of the TAA-treated mice after transplantation of hE-MSCs, tissue sections of the liver were stained with an antibody against TIF1γ and CRBP1 which is a hepatic stellate cell marker 14 days after transplantation. Specifically, paraffin in paraffin tissue sections of TAA-treated mouse livers experiencing liver fibrosis was peeled off by xylene, and the tissue sections were hydrated with alcohol. After antigens were recovered by applying heat to the tissue sections in a citric acid buffer (DAKO, Glostrup, Denmark), non-specific binding sites were blocked with 1% bovine serum albumin of PBS containing 0.01% Triton X-100. According to the used antibody, permeabilization was selectively performed in PBS of 0.1% Triton X-100 for 10 minutes before the blocking. Thereafter, the tissue sections were cultured at 4° C. overnight using primary antibodies such as anti-TIF1γ (1:1000, Abcam, Cambridge, UK), anti-cellular retinol-binding protein 1 (CRBP1, 1:100; Santa Cruz Biotechnology, Santa Cruz, Calif., USA), anti-α-SMA (1:800; Sigma-Aldrich), anti-hepatocyte (Hepatocyte Paraffin-1; Hep Par-1) (1:300, DAKO) or anti-HGF (1:100; Abcam). After washing, the tissue sections were cultured with Alexa Fluor-conjugated fluorescent antibodies (Invitrogen) at room temperature for 2 hours, and then washed with PBS, and fluorescence was fixed using 4′,6-diamidino-2-phenylindole (DAPI; IHC World, Woodstock, Md., USA). Images were obtained using a confocal microscope (Carl Zeiss LSM710, Gottingen, Germany). In addition, a quantitative analysis was performed by the method described in Example 2-1.

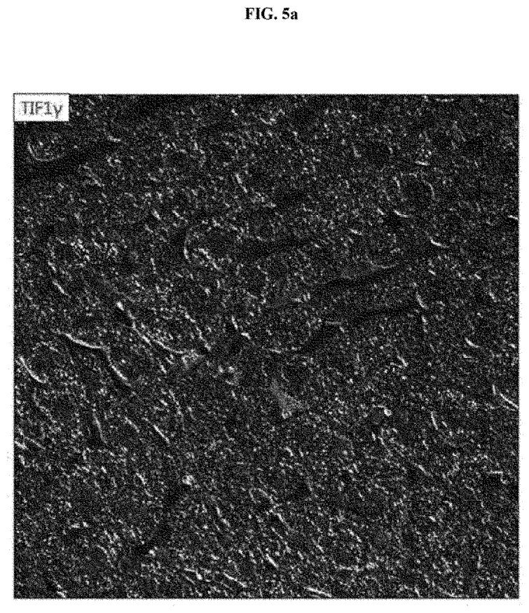

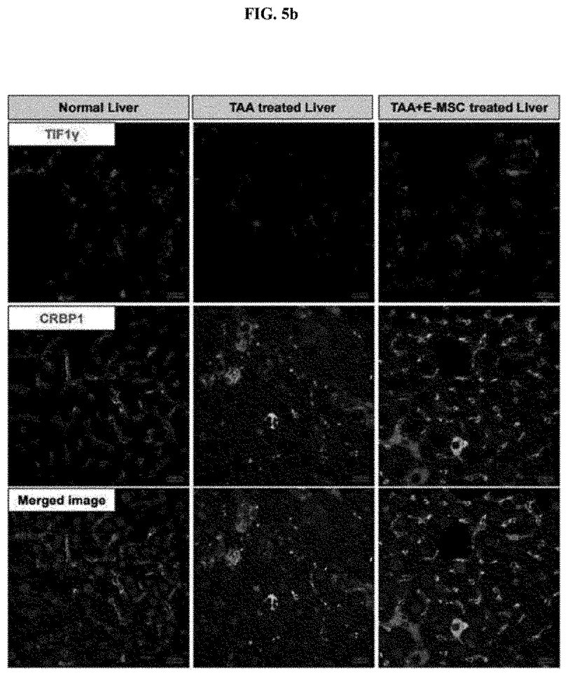

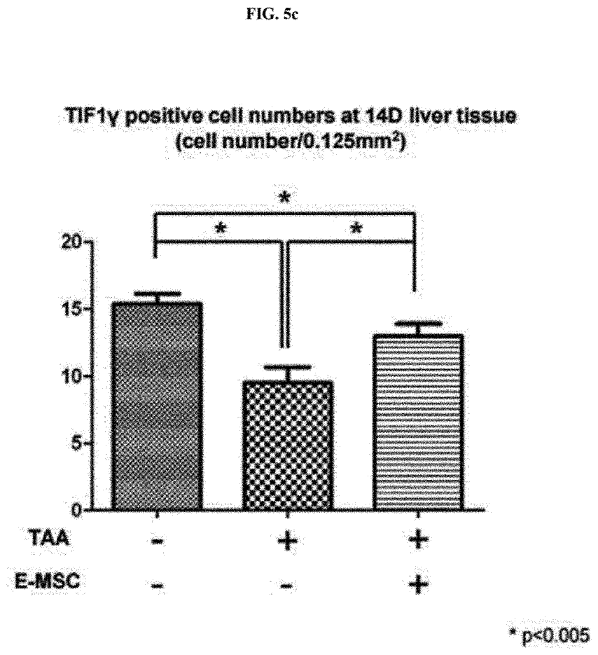

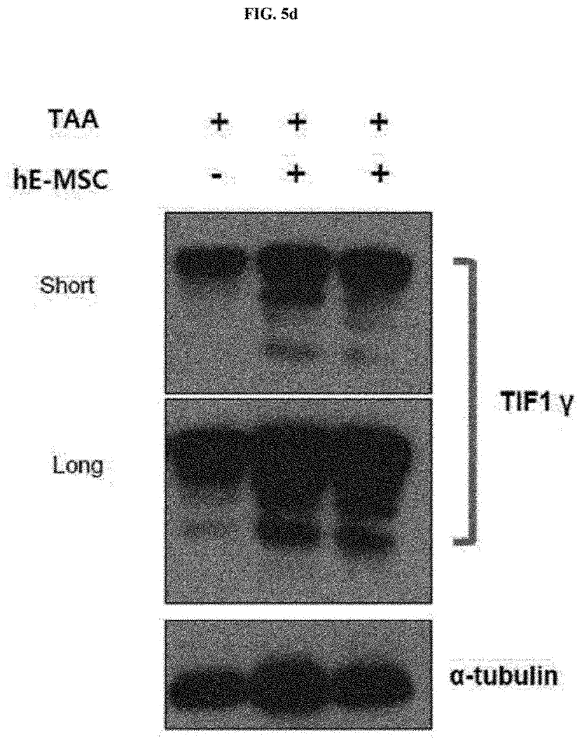

As a result, as illustrated in FIG. 5A, first, it was confirmed that in the normal liver, positive cells were discovered in the perisinusoidal space (or space of Disse), and TIF1γ was expressed, and as illustrated in FIG. 5B, it was confirmed that in the TAA-treated liver, the expression of CRBP1 and TIF1γ was restored 14 days after transplantation of hE-MSCs.

Further, as illustrated in FIG. 5C, as a result of quantitatively analyzing TIF1γ positive cell numbers 14 days after transplantation of hE-MSCs in the TAA-treated liver, it was confirmed that as a result of transplantation of hE-MSCs, the expression of TIF1γ was remarkably increased as compared to the control and the TAA-treated mice.

6-2. Western Blot Analysis

In order to confirm the expression of TIF1γ according to the transplantation of hE-MSCs in the TAA-treated mouse liver in Example 6-1, Western blot analysis was performed by the method described in Example 3-3.

As a result, as illustrated in FIG. 5D, it was shown that the expression of TIF1γ in the TAA-treated liver was upregulated by transplantation of hE-MSCs. This shows that TIF1γ is a potential anti-fibrosis factor, expressed in hepatic stellate cells, downregulated by a liver fibrosis precursor signal such as TAA and TGFβ1, and upregulated by an anti-fibrosis stimulus such as transplantation of hE-MSCs.

### Example 7

### Confirmation of Secretion of Hepatic Stellate Cells (HSCs) and Secretion of Human Hepatocyte Growth Ffactor (hHGF) According to

### Transplantation of Human Embryonic Stem Cell-Derived Mesenchymal Stem Cells

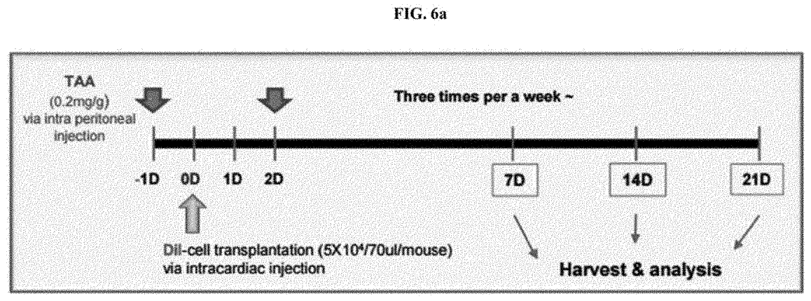

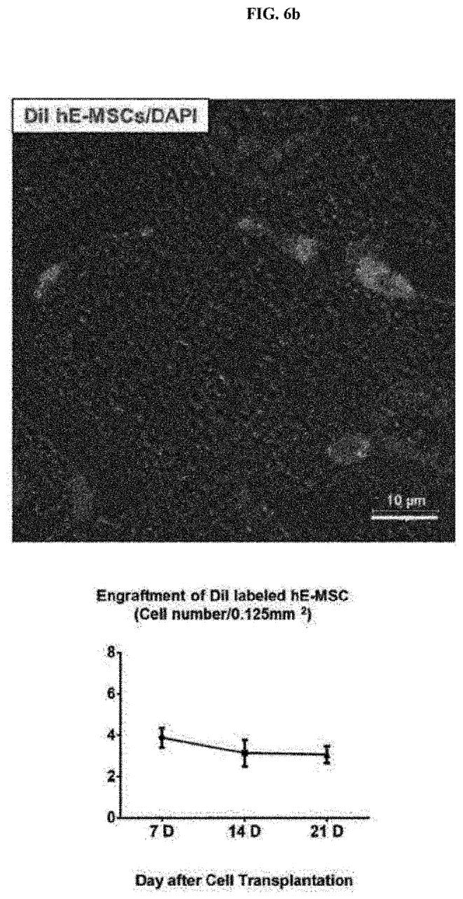

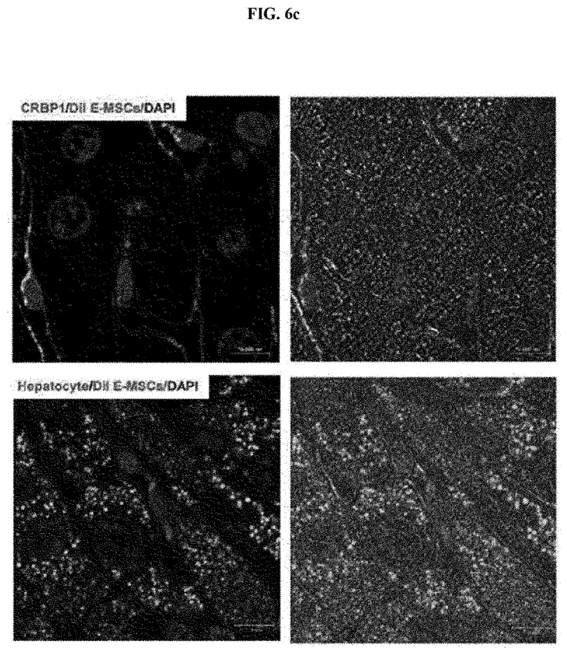

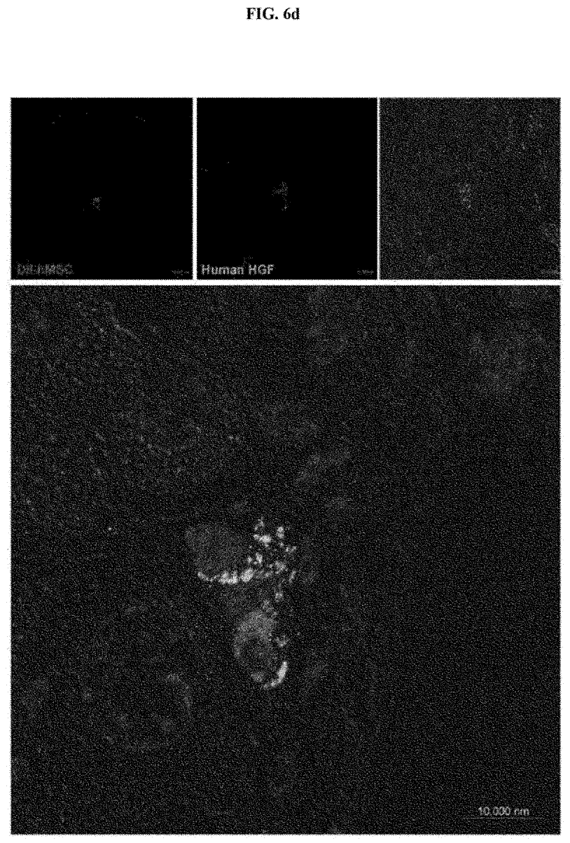

As illustrated in FIG. 6A, in order to track hE-MSCs, hE-MSCs were labeled with a fluorescent dye (DiI), and an immunohistochemistry assay was performed 7, 14, and 21 days after transplantation of hE-MSCs into the TAA-treated liver by the method described in Example 6-1. Further, immunofluorescence staining was performed using CRBP1 and hepatocyte antibodies, and the secretion of hepatocyte growth factor from transplanted cells was evaluated using human hepatocyte growth factor-specific antibodies.

As a result, as illustrated in FIG. 6B, although a slight decrease in fluorescent cells was exhibited, fluorescence was still observed even 21 days later.

In addition, as illustrated in FIG. 6C, DiI-positive cells were stained with CRBP1 without reacting with hepatocyte antibodies. Even though the observation result may not be confirmed by in vivo functional analysis, the differentiation of hE-MSCs into hepatic stellate cells is exhibited.

Likewise, as illustrated in FIG. 6D, as a result of evaluating the secretion of hepatocyte growth factor from transplanted cells using human hepatocyte growth factor-specific antibodies, human hepatocyte growth factor (hHGF) secreted by DiI positive cells was detected. The staining of the human hepatocyte growth factor was observed in neighboring adjacent cells rather than in DiI positive cells. From these results, it is expected that in the TAA-treated mouse liver, some hE-MSCs survived, differentiated into hepatic stellate cells, and were able to secrete paracrine HGF.

### Example 8

### Confirmation of TIF1γ Inhibition Effect in Human Cirrhotic Liver

In order to confirm whether the experimental results in the mouse model could also be applied to humans, the immunochemical analysis described in Example 2-2 was performed on human liver tissue (purchased from SuperBioChip Lab. Seoul, Korea). The degree of liver fibrosis was expressed as F0 (no fibrosis) to F4 (cirrhosis) or 0 (no fibrosis) to 6 (cirrhosis) according to the METAVIR criteria or ISHAK stages (Standish, 2006), respectively.

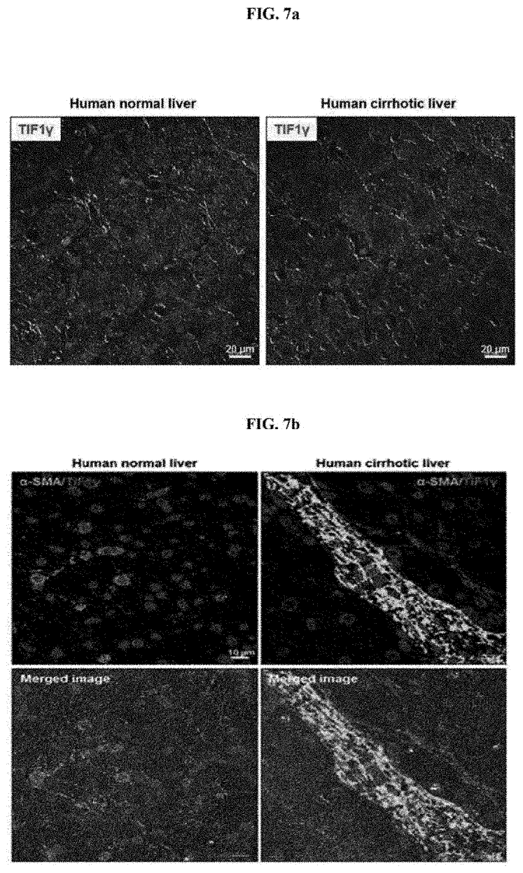

As a result, as illustrated in FIG. 7A, it was observed that in the human cirrhotic liver (ISHAK 6/METAVIR F4), the expression of TIF1γ was decreased, and as illustrated in FIG. 7B, the expression of α-SMA was increased. These results suggest that TIF1γ is an anti-fibrosis factor which plays an important role in maintaining the health of the liver and can be used to develop a new therapeutic approach capable of restoring and preventing liver fibrosis.

The above-described description of the present invention is provided for illustrative purposes, and one skilled in the art to which the present invention pertains will understand that the present invention can be easily modified into other specific forms without changing the technical spirit or essential features of the present invention. Therefore, it should be understood that the above-described Examples are illustrative only in all aspects and are not restrictive.

### INDUSTRIAL APPLICABILITY

The pharmaceutical composition for preventing or treating liver fibrosis or cirrhosis, comprising an expression or activity enhancer of TIF1γ as an active ingredient, according to the present invention, inhibits the activity of hepatic stellate cells (HSCs) and decreases the expression of α-SMA proteins or the secretion of collagen Type I, thereby being expected to be useful as a prophylactic or therapeutic agent for liver fibrosis or cirrhosis, and in addition, it is expected that the composition of the present invention can be utilized to screen an agent for liver fibrosis or cirrhosis.

1.-5. (canceled)

6. A method for screening a candidate material for preventing or treating liver fibrosis or cirrhosis, comprising steps of (1) treating cells or tissues harvested from a patient with liver fibrosis or cirrhosis with a test material and culturing the treated cells or tissues; (2) measuring an expression level of TIF1γ in a cell or tissue culture solution of Step (1); and (3) selecting a candidate material which increases the expression of TIF1γ as compared to a control which is not treated with the test material.

7. The method of claim 6, wherein the test material is a synthetic compound, a microbial culture solution or extract, a synthetic peptide, a nucleic acid, a protein, an antibody, an aptamer, or a natural extract.

8. A method for preventing or treating liver fibrosis or cirrhosis, comprising administrating to a subject, a pharmaceutical composition comprising an expression enhancer or activity inducer of transcriptional intermediary factor 1 gamma (TIF1γ ) as an active ingredient.

9. (canceled)

10. The method of claim 8, wherein the expression enhancer or activity inducer of TIF1γ is a human embryonic stem cell-derived mesenchymal stem cell.

11. The method of claim 8, wherein the expression enhancer or activity inducer of TIF1γ is hepatocyte growth factor (HGF), a histone deacetylase (HDAC) inhibitor, a transforming growth factor beta (TGF-β) signal inhibitor, or an epithelial-mesenchymal transition (EMT) inhibitor.

12. The method of claim 8, wherein the composition downregulates the expression of α-smooth muscle actin (α-SMA) proteins.

13. The method of claim 8, wherein the composition decreases the secretion of collagen Type I.
